(19) United States
(12) Patent Application Publication
(10) Pub. No.: US 2009/0247853 A1
(43) Pub. Date: Oct. 1, 2009

(54) Non-Invasive Total Hemoglobin Measurement by Spectral Optical Coherence Tomography

(75) Inventor: Martin Debreczeny, Danville, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/413,670

(22) Filed: Mar. 30, 2009

Related U.S. Application Data
(60) Provisional application No. 61/040,815, filed on Mar. 31, 2008.

(51) Int. Cl. A61B 5/1455

(52) U.S. Cl. 600/328

(57) ABSTRACT

Spectral Optical Coherence Tomography (“SOCT”) utilizes Optical Coherence Tomography (“OCT”) to depth-target two or more optical measurements within a blood vessel. OCT achieves depth resolution by the use of optical interferometry. As the path length of the reference arm of the interferometer is varied, the penetration depth at which maximum interference occurs (zero phase difference) in the sample is correspondingly increased. Depth resolution in the range of 10 μm to 100 μm enables measurements that may be made within more narrow spectral regions (in the range of 1 to 50 nm) in multiple regions of the visible and near infrared spectrum. In one embodiment the light source is configured for three spectral regions centered at 805 nm, 980 nm, and 1050 nm. By comparing the OCT signal at these different spectral positions, the absorption due to tissue and blood analytes may be measured.

### CROSS-REFERENCE TO RELATED APPLICATION

This claims the benefit of U.S. Provisional Patent Application No. 61/040,815, filed Mar. 31, 2008, which is hereby incorporated by reference herein in its entirety.

### BACKGROUND

This invention relates to optical measurement of total hemoglobin, and more particularly to in vivo non-invasive measurement of total hemoglobin.

In vivo non-invasive optical measurement of total hemoglobin (or closely-related hematocrit) is complicated by the need for light to travel through the skin before passing into the vasculature, where the desired measurement is to be made. Proposed methods of overcoming this obstacle have taken an approach in a manner analogous to pulse oximetry: by measurement of the photoplethysmograph due to arterial pulsation. In pulse oximetry, the amplitude of the plethysmograph at two different wavelengths (e.g., 660 nm and 890 nm) is ratioed and related to arterial oxygen saturation. Proposed methods directed toward non-invasive total hemoglobin measurement have suggested extension of this idea to include wavelengths at which total hemoglobin (e.g., 805 nm) and water (e.g., 1310 nm) absorption can be estimated. In this way, ideally, a ratio of plethysmographic amplitudes at two wavelengths could be related to the total hemoglobin concentration in the blood. The feasibility of this approach has been demonstrated using whole blood in vitro. However, in vivo studies have so been far less accurate and not reliable. One likely reason for the poor performance of plethysmographic total hemoglobin estimation in vivo is due to the fact that water, unlike hemoglobin, is not located primarily within the vasculature, but is instead present in high concentration in all living tissues and fluids. As a result, the plethysmographic signal at water-absorbing wavelengths is probably indicative of the difference between the water within the vasculature and that in the surrounding tissues.

### SUMMARY

This Summary is provided to introduce in a simplified form a selection of concepts that are further described below in the Detailed Description. This Summary is not intended to identify key features or essential features of the claimed subject matter, nor is it intended to be used to limit the scope of the claimed subject matter.

The present approach overcomes the limitations described above by depth-targeting the optical measurement within a blood vessel by using Optical Coherence Tomography (“OCT”). This depth-targeting optical measurement technique is referred to as Spectral Optical Coherence Tomography (“SOCT”). OCT achieves depth resolution by the use of optical interferometry. As the path length of the reference arm of the interferometer is varied, the penetration depth at which maximum interference occurs (zero phase difference) in the sample is correspondingly increased. OCT is capable of achieving two μm depth resolution at penetrations depths up to several mm. However, such high depth resolution requires the use of a very broad spectral source (100 nm or more). In the present approach, lower depth resolution may be tolerated (in the range of 10 μm to 100 μm), so that measurements may be made within more narrow spectral regions (in the range of 1 to 50 nm) in multiple regions of the visible and near infrared spectrum. By comparing the OCT signal at these different spectral positions, the absorption due to tissue and blood analytes may be measured. The light source emits light centered on three different wavelengths enabling the measurement of total hemoglobin and water concentrations within the blood vessel, independent of the oxygen saturation of the hemoglobin.

### BRIEF DESCRIPTION OF THE DRAWINGS

FIG. 1 shows a schematic/block diagram of an embodiment of spectral optical coherence tomography of the present invention.

FIG. 2 shows a block flow diagram of an embodiment of spectral optical coherence tomography of the present invention.

### DETAILED DESCRIPTION

Optical coherence tomography is an interferometric, non-invasive optical tomographic imaging technique offering millimeter penetration (approximately two to three mm in tissue) with micrometer-scale axial and lateral resolution. OCT is capable of achieving sub-micrometer axial resolution due to wide bandwidth light sources (sources emitting wavelengths over a ˜100 nm range).

OCT is based on low-coherence interferometry. In conventional interferometry with long coherence length (laser interferometry), interference of light occurs over a distance of meters. In OCT, this interference is shortened to a distance of micrometers, thanks to the use of broadband light sources (sources that can emit light over a broad range of frequencies). Light with broad bandwidths can be generated by using super luminescent diodes (super-bright LEDs) or lasers with extremely short pulses (femto-second lasers). White light is also a broadband source with lower powers.

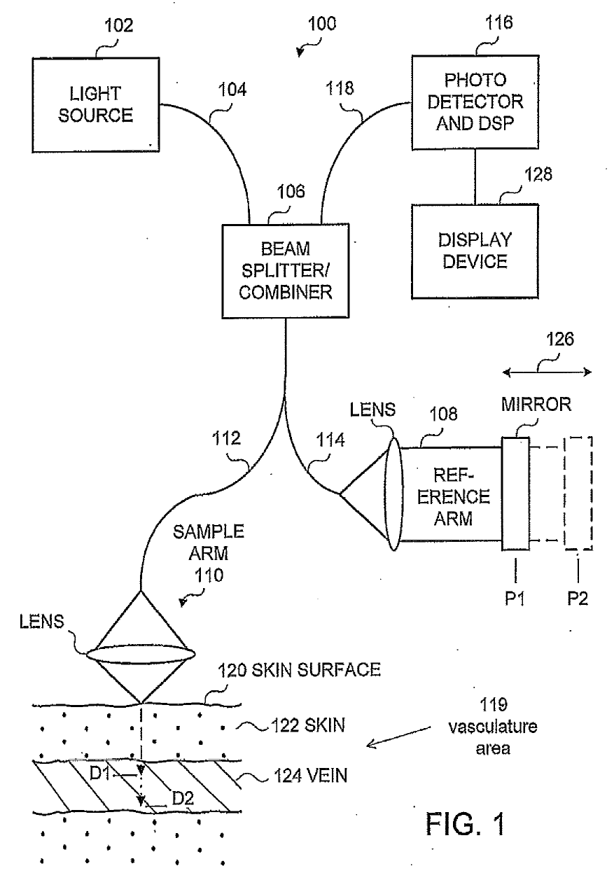

Referring now to the Figures, in which like reference numerals refer to structurally and/or functionally similar elements thereof, FIG. 1 shows a schematic/block diagram of an embodiment of Spectral Optical Coherence Tomography (“SOCT”) of the present invention. Referring now to FIG. 1, the optical setup for SOCT consists of an Interferometer 100 (which may be a Michelson type) with a low coherence, broad bandwidth light source. Light from Light Source 102 is carried through Fiber Optic Cable 104 to Beam Splitter 106. The light is split into and recombined from a Reference Arm 108 and a Sample Arm 110 via Fiber Optic Cables 112 and 114. Sample Arm 110 is placed over Skin Surface 120 of Skin 122 in a location above Vein 124. Photo Detector and Digital Signal Processor 116 receives light from measurements taken via Fiber Optic Cable 118. Display Device 128 displays the results of the measurements taken.

In one embodiment of taking SOCT measurements, Light Source 102 is configured for three spectral regions centered at 805 nm, 980 nm, and 1050 nm wavelengths. The sensor of Sample Arm 110 is positioned over a superficial vascular structure, such as Skin 122 with Vein 124. Reference Arm 108 of Interferometer 100 is free to move back and forth in the direction indicated by Arrow 126 and is positioned so as to achieve maximum interference with Sample Arm 110 of Interferometer 100 when the sample beam is located in the interior of the vascular structure (Vein 124). At this depth, measurements at the three wavelengths may be combined to determine the relative concentrations of total hemoglobin and water within the vessel, independent of the oxygen saturation of the hemoglobin.

The blood absorption coefficient at 805 nm only depends on hematocrit. The blood absorption coefficient at 980 nm and 1050 nm depends on hematocrit and water. Therefore, in addition to determining the relative concentration of total hemoglobin and water within the vessel, the oxygen saturation can also be determined from the measurements at the three wavelengths.

The present invention takes advantage of the known attenuation of the OCT measurement as a function of sample depth. In order to compensate for this attenuation, multiple SOCT measurements may be compared at more than one depth. For example, by comparing SOCT measurements at two or more depths within the same vessel, the effect of the intervening tissue may be largely removed by subtracting the two (or more) signals from each other. FIG. 1 shows the mirror of Reference Arm 108 in a first position P1, which corresponds to a measurement taken at depth D1. Reference Arm 108 is shown in phantom in a second position P2, which corresponds to a measurement taken at depth D2. The difference signal would then reflect only changes in signal attenuation due to blood.

The multiple measurements would preferably be made within a relatively large vessel (>50 μm), under relatively low flow (such as in a vein), in order to maintain the homogeneity of the blood. In small vessels under high flow, red blood cells tend to be more concentrated at the center of the vessel. Another benefit in measuring blood within a large vessel is that it will likely be more reflective of the systemic total hemoglobin. This is due to the fact that total hemoglobin in the capillaries is typically significantly lower and less variant to hemoglobin variations than in larger vessels.

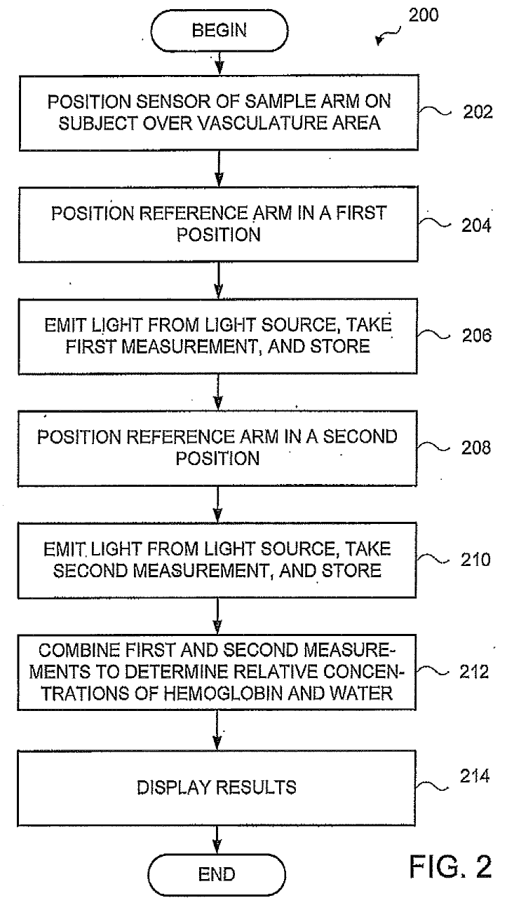

FIG. 2 shows a block flow diagram of an embodiment of a method for spectral optical coherence tomography of the present invention. Referring now to FIG. 2, the method 200 begins in step 202 with positioning the sensor of a sample arm of an interferometer, such as Sample Arm 110 of Interferometer 100 shown in FIG. 1, on the skin of a subject, human or animal, over a superficial vasculature area. In step 204 the reference arm of the interferometer, such as Reference Arm 108 of Interferometer 100 is positioned so that the sample beam is located in the interior of the vascular structure at a first depth to achieve maximum interference with Sample Arm 110, such as depth D1 and position P1 shown in FIG. 1. Light is emitted from a light source that is configured for three spectral regions centered at 805 nm, 980 nm, and 1050 nm, such as Light Source 102 of Interferometer 100. A first measurement of the returned light from the sample arm and the reference arm is then taken in step 206 with a photo detector and digital signal processor, such as Photo Detector and Digital Signal Processor 116 of Interferometer 100. The measurement is stored for further processing.

In step 208, with the sensor in the same position, the reference arm of the interferometer is moved to a second position so that the sample beam is located in the interior of the vascular structure at a second depth to achieve maximum interference with the sample arm, such as depth D2 and position P2 shown in FIG. 1. Light is emitted from the light source that is configured for three spectral regions and a second measurement is taken in step 210. The second measurement is detected with the photo detector and processed and stored by the digital signal processor. The two measurements are combined and the relative concentrations of total hemoglobin and water within the vessel, independent of the oxygen saturation of the hemoglobin, may then be displayed on a display device, such as Display Device 128. The method then ends. The method may of course be repeated with the sensor at the same location, or moved to a different location over the same vasculature area, or moved to a new vasculature area.

Having described the present invention, it will be understood by those skilled in the art that many changes in construction and circuitry and widely differing embodiments and applications of the invention will suggest themselves without departing from the scope of the present invention. It will further be understood that the steps described and claimed herein are not limited in time to the order disclosed.

What is claimed is:

1. A method for determining total hemoglobin in vivo, the method comprising the steps of:
(a) positioning a sensor of a sample arm of an interferometer on the skin of a subject over a vasculature area;
(b) positioning a reference arm of the interferometer in a first position to achieve maximum interference with the sample arm that corresponds to a first depth within the vasculature area;
(c) emitting light a first time from a first light source having three spectral regions;
(d) measuring the light returned from the sample arm and the reference arm;
(e) storing the measured light as a first measurement;
(f) positioning the reference arm of the interferometer in a second position to achieve maximum interference with the sample arm that corresponds to a second depth within the vasculature area;
(g) emitting light a second time from the light source having three spectral regions;
(h) measuring the light returned from the sample arm and the reference arm;
(i) storing the measured light as a second measurement; and
(j) combining the first measurement and the second measurement to determine a relative concentration of hemoglobin.

2. The method according to claim 1 wherein said emitting light steps (c) and (g) further comprise the steps of:
emitting light in a first spectral region centered on 805 nm wavelength;
emitting light in a second spectral region centered on 980 nm wavelength; and
emitting light in a third spectral region centered on 1050 nm wavelength.

3. The method according to claim 1 further comprising the step of:
displaying the relative concentration of hemoglobin on a display device.

4. The method according to claim 1 further comprising the step of:
combining the first measurement and the second measurement to determine a relative concentration of water.

5. The method according to claim 4 further comprising the step of:
displaying the relative concentration of water on a display device.

6. The method according to claim 1 wherein said combining step (j) is independent of the oxygen saturation of the hemoglobin.

7. The method according to claim 1 wherein said measuring steps (d) and (h) further comprise the steps of:
capturing the light returned from the sample arm and the reference arm with a photo detector;
processing the output of the photo detector with a digital signal processor.

8. A system for determining total hemoglobin in vivo, the system comprising:
an interferometer, the interferometer further comprising:
a sample arm having a sensor that is positioned on the skin of a subject over a vasculature area;
a reference arm moved to a first position to achieve maximum interference with the sample arm that corresponds to a first depth within the vasculature area;
a light source capable of emitting light in three spectral regions;
a beam splitter/combiner capable of splitting and combining the emitted light;
a photo detector capable of capturing light returned from the sample arm and the reference arm; and
a digital signal processor capable of processing the output of the photo detector;
wherein light is emitted a first time from the light source with the reference arm in a first position to achieve maximum interference with the sample arm that corresponds to a first depth within the vasculature area, and light is emitted a second time from the light source with the reference arm in a second position to achieve maximum interference with the sample arm that corresponds to a second depth within the vasculature area, wherein a first measurement from the first light emission and a second measurement from the second light emission processed by the digital signal processor are combined to determine the relative concentration of hemoglobin.

9. The system according to claim 8 wherein said light source further comprises:
a first diode capable of emitting light in a first spectral region centered on 805 nm wavelength;
a second diode capable of emitting light in a second spectral region centered on 980 nm wavelength; and
a third diode capable of emitting light in a third spectral region centered on 1050 nm wavelength.

10. The system according to claim 8 wherein said first further comprising:
a display device capable of displaying the relative concentration of hemoglobin.

11. The system according to claim 8 wherein the first and second measurements are combined by the digital signal processor to determine a relative concentration of water.

12. A method of estimating total hemoglobin in a patient's blood comprising:
transmitting light into skin of the patient;
measuring first intensities of at least three different wavelengths of light reflected from a first depth in the skin;
measuring second intensities of at least three different wavelengths of light reflected from a second depth in the skin; and
estimating the total hemoglobin in the patient's blood based on a comparison of the first intensities to the second intensities.

13. The method of claim 12 wherein the at least three different wavelengths include at least one wavelength selected based on its absorption by water in the skin.

14. The method of claim 12 wherein the at least three different wavelengths include two wavelengths that can be compared to determine the oxygen saturation of the patients blood.

15. The method of claim 12 wherein the at least three different wavelengths include a first wavelength of about 805 nm, a second wavelength of about 980 nm, and a third wavelength of about 1050 nm.
